(19) United States
(12) Patent Application Publication
(10) Pub. No.: US 2008/0213394 A1
(43) Pub. Date: Sep. 4, 2008

(54) Polymer-Based Antimicrobial Agents, Methods of Making Said Agents, and Products Incorporating Said Agents

(76) Inventors: Louis J. Tullo, Hewlett, NY (US); Leonard Pinchuk, Miami, FL (US); Ashraf A. Ismail, Westmount (CA); Orley R. Pinchuk, Montreal West (CA); David Pinchuk, Montreal West (CA)

Correspondence Address: GORDON & JACOBSON, P.C., 60 LONG RIDGE ROAD, SUITE 407, STAMFORD, CT, 06902, US

(21) Appl. No.: 11/908,298

(22) PCT Filed: Mar. 11, 2005
(86) PCT No.: PCT/US2005/008360

§ 371 Date: Sep. 11, 2007

(51) Int. Cl. A61K 33/38; A01P 1/00; A01N 59/16

(52) U.S. Cl. 424/618

(57) ABSTRACT

An antimicrobial agent includes a metal ion in a hydrophilic polymer binder or carrier. The metal ion is preferably a silver ion and the hydrophilic polymer preferably comprises a sulfonated polyurethane or sulfonated polystyrene. According to a method of the invention, the antimicrobial agent is dissolved in dimethyl acetamide DMA, applied to paper by spraying, squeegee or the like and dried in an oven to flash off the solvent. The antimicrobial agent can be applied to other products by spraying and/or dipping and then drying to flash off solvent. According to another embodiment of the invention, the antimicrobial agent includes a water soluble polymer, at least one organic acid (e.g., one or more carboxylic acids such as acetic acid, formic acid, citric acid, malefic acid, ascorbic acid, salicyclic acid), and oligodynamic metal ions which react with counter-ions of the polymer such that the metal ions are bound to corresponding counter-ions, and the polymer controls a sustained release of said metal ion. The agent may also include a non-organic acid. (preferably boric acid and/or dictylborate). The water soluble polymer is preferably a sulfonated polymer (e.g., a sulfonated polyurethane, a sulfonated polystyrene, or a mixture thereof).

This application is a continuation-in-part of U.S. patent application Ser. No. 10/138,160 filed on May 2, 2002, which is herein incorporated by reference in its entirety.

### BACKGROUND OF THE INVENTION

1. Field of the Invention

The invention relates to antimicrobial agents, products incorporating such agents, and methods of making such products. More particularly, the invention relates to polymer-based antimicrobial agents.

2. State of the Art

Silver and silver salts are commonly used as antimicrobial agents. An early medicinal use of silver was the application of aqueous silver nitrate solutions to prevent eye infection in newborn babies. Silver salts, colloids, and complexes have also been used to prevent and to control infection. Other metals, such as gold, zinc, copper, and cerium, have also been found to possess antimicrobial properties, both alone and in combination with silver. These and other metals have been shown to provide antimicrobial behavior even in minute quantities, a property referred to as “oligodynamic.”

U.S. Pat. No. 6,306,419 to Vachon et al. discloses a polymer-based coating comprising a styrene sulfonate polymer with a silver metal incorporated therein. The styrene sulfonate polymer is prepared by reacting an acetyl sulfate sulfonation agent with a styrene copolymer in 1,2-dichloroethane (DCE). The coating is hydrophilic such that it retains a relatively large amount of water or water-containing fluid. There are several disadvantages to this composition. One such disadvantage is that larger quantities of the silver metal are required to provide effective antimicrobial activity. A second disadvantage is that a solvent (e.g. DCE) is required to prepare the polymer matrix. Such solvents are typically hazardous because of their reactive nature and thus require special care in handling and disposing of such solvents, which limits the widespread acceptance of such antimicrobial polymers in many applications.

### SUMMARY OF THE INVENTION

It is therefore an object of the invention to provide a hydrophilic polymer-based antimicrobial agent that does not require relatively large quantities of the metal in order to provide effective antimicrobial activity.

It is also an object of the invention to provide a hydrophilic polymer-based antimicrobial agent that is readily soluble in a water solution.

It is another object of the invention to provide an antimicrobial agent which can be incorporated in paper products.

It is another object of the invention to provide methods of incorporating an antimicrobial agent, which is capable of killing anthrax on contact, with other products including paper products and certain medical products.

In accord with these objects which will be discussed in detail below, the antimicrobial agent of the present invention includes a metal ion in a hydrophilic polymer binder or carrier. The metal ion is preferably a silver ion and the hydrophilic polymer preferably comprises a sulfonated polyurethane or sulfonated polystyrene.

According to a method of the invention, the antimicrobial agent is dissolved in dimethyl acetamide DMA, applied to paper by spraying, squeegee or the like and dried in an oven to flash off the solvent. The antimicrobial agent can be applied to other products by spraying and/or dipping and then drying to flash off solvent.

Paper coated with the antimicrobial agent of the invention was tested by NAMSA (Atlanta, Ga.) for antimicrobial activity using the Dow 923 “Shake-flask” test. After one hour 99.94% (the upper limit of the test equipment) of all bacteria were killed.

According to another embodiment of the invention, the antimicrobial agent includes a water soluble polymer, at least one organic acid (e.g., one or more carboxylic acids such as acetic acid, formic acid, citric acid, maleic acid, ascorbic acid, salicyclic acid), and oligodynamic metal ions which react with counter-ions of the polymer such that the metal ions are bound to corresponding counter-ions, and the polymer controls a sustained release of said metal ion. The agent may also include a non-organic acid (preferably boric acid and/or dictylborate). The water soluble polymer is preferably a sulfonated polymer (e.g., a sulfonated polyurethane, a sulfonated polystyrene, or a mixture thereof).

Additional objects and advantages of the invention will become apparent to those skilled in the art upon reference to the detailed description.

### DETAILED DESCRIPTION OF THE PREFERRED EMBODIMENTS

The antimicrobial agents according to the invention utilize a metal ion in conjunction with a hydrophilic polymer. The metallic ions, derived from metals such as Ag, Au, Pt, Pd, Ir (i.e., the noble metals), Cu Sn, Sb, Bi and Zn, as well as many heavy metals, are effective antimicrobials.

Metallic antimicrobials function by releasing metal ions into the microbe. The released ions react with protein and other anions (negative charged species) in the microbe and render the protein insoluble and thereby inactive. Inactive protein perturbs cellular function, disrupts membranes and prevents the normal activity and reproduction of DNA thereby essentially killing the microorganism. In order for antimicrobials to release metal ions into the microbe, the microbe must be in fluidic contact with the metal ion, i.e., they must both be in the same water medium. In addition, the metal ion must release from the substrate it is attached to, diffuse out to the microbe, penetrate the membrane of the microbe, seek protein, bind to it and then precipitate it. Importantly, most of the more deadly microbes, such as anthrax are not water-containing. The anthrax spore is essentially dry and inert to environmental conditions due to its durable membrane and lack of moisture within the membrane.

Of the metal ions mentioned above, silver ion (Ag+) is perhaps the best known metal ion antimicrobial due to its unusually good bioactivity at low concentrations. This bioactivity of silver is known as oligodynamic action. However, Ag+ is not stable. In the presence of light, Ag+ converts to Ag metal. This instability is a benefit for the photography industry. Ag+ is clear, Ag metal is opaque-black. For these reasons, Ag+ is not a likely candidate for an antimicrobial treatment of paper. Paper treated with Ag+ will turn black when exposed to light and will no longer have any antimicrobial effect. Even if the paper were not exposed to light, if Ag+ is released from the paper too rapidly, the Ag+ reservoir will be depleted, excess Ag+ will convert to its metal form and the antimicrobial activity will be compromised. If the Ag+ is released too slowly, however, it may not be present in sufficient quantity to be effective.

Despite the disadvantages of Ag+, the present invention has found a way to overcome these disadvantages and the disadvantage of metal ions in general (that they need water to work as antimicrobials) particularly with regard to very dry microbes such as anthrax spores.

According to the invention Ag+ is bound to a substrate which releases it at just the correct rate, protects it from light, and also hydrates easily with water, or more preferably remains wet for a long time. The substrate preferably contains wetting groups that are more than simple water absorbing groups; rather these groups essentially suck in water and bind the water to the surface somewhat permanently.

The substrate of the invention is easily rendered into a lacquer which can be applied to paper without mottling, softening, wetting, or the like. In addition, the solvents used to form the lacquer are preferably non-toxic, non-flammable, non-carcinogenic, non-mutagenic, etc.

An antimicrobial according to the invention therefore preferably includes a polymer or molecular substance that has pendant hydrophilic groups that include, sulfates, carboxylic acids, amines, hydroxyls, nitrates, phosphates, or in general, any functional group soluble in water. More preferably, the hydrophilic group is also capable of binding with an oligodynamic metal ion, such as Ag+ or Zn²⁺. Negatively charged hydrophilic groups such as sulfates, phosphates, nitrates, carboxylates and the like, are therefore preferred.

Polymers useful for the solids content of the lacquer include polyurethane, polyamines, cellulose, cellulose acetate, triacetate, polyester, hydrogels, polyolefins, and any other polymer capable of dissolving or dispersing in a solvent. Chemical substances can include surfactants, silane coupling agents, etc., that have tails that are hydrophobic and head groups that are hydrophilic; the hydrophilic heads include the pendants mentioned above. Included in this list are the aforementioned polymers and chemical substances that have been further modified to increase solubility, increase their reactivity towards metal ions and modified further to modulate their activity in regard to the sustained release of the metal ions.

The antimicrobial agent of the invention is illustrated in nine examples.

### Example 1

A polymer solution is made by dissolving 10 g of an aromatic polyether urethane such as Dow Chemical's Pellethane™ 2363 75D in 18 g dimethyl acetamide (DMA) and 72 g tetrahydrofuran (THF) at 70° C. with mixing for 3 hours.

The polyurethane is sulfonated and rendered hydrophilic by adding 21 ml of acetic anhydride and 12.5 ml of concentrated sulfuric acid to the polyurethane solution while it is being vigorously mixed. After the exothermic reaction subsides, the hydrophilic urethane is poured into a blender filled with water where the polyurethane is precipitated and chopped into small particles under agitation. The particulate slurry is poured through a wire sieve to remove the particles and rinsed repeatedly with water until the pH of the solution is between 4 and 8. The precipitated sulfonated polyurethane is then dried for 3 hours in an oven at 70° C.

The dried sulfonated polyurethane (10 g) is then redissolved in DMA. Films cast from this solution dry to clear and turn white opaque upon soaking in water for a few minutes. The opaque white transition is typical of polymers that absorb water thereby confirming that the polyurethane thus formed is hydrophilic.

The sulfonated polyurethane (10 g dissolved in 90 g DMA) solution is then reacted with silver nitrate by adding 0.2 g (2% by weight of polymer) of silver nitrate to the sulfonated polyurethane solution. The solution turns milky white after the addition thereby indicating a reaction between the sulfate groups and the silver nitrate.

The polyurethane with the silver sulfate groups is then squeegeed onto paper or the like and dried in an oven at 70° C. for 10 minutes to flash off the solvent. The dried coated paper is tested for elutable silver by placing a section of the coated paper under an ultraviolet lamp, adding a drop of water to a section of the paper and exposing the paper with the drop of water to the UV light for 15 to 20 minutes. It can easily be observed that the drop of water turns gray as the silver ion migrates from the substrate and is converted to silver metal by the ultraviolet light. Areas around the drop of water do not significantly change color.

Coated sections of paper produced according to this example were tested for antimicrobial activity by NAMSA using the Dow 923 “Shake-flask” test which involves shaking the sample in a flask with staphylococcus aureus bacteria for 1 hour and then for 24 hours and measuring the amount of bacteria killed. Results indicate that at one hour, 99.94%, or essentially all, of the bacteria were killed. The results at 24 hours were the same which suggests that there were no bacteria remaining to be killed, i.e. that 99.94% is the upper limit of the testing equipment.

The antimicrobial of this example provides a sustained release of Ag+ due to the sulfate counter-ions. The Ag+ is released at a rate sufficient to kill bacteria on contact but slow enough that the antimicrobial activity is maintained over a long time. The effective duration of the coating is dependent upon many factors, such as the thickness of the coating, the ratio of silver to polymer, and the degree of hydration of the system. The efficacy of typical coatings can last years depending upon the particular parameters.

### Example 2

A polymer solution is made by dissolving 10 g of an aromatic polyether urethane such as Dow Chemical's Pellethane™ 2363 75D in 18 g dimethyl acetamide (DMA) and 72 g tetrahydrofuran (THF) at 70° C. with mixing for 3 hours. Silver sulfadiazine in the amount of 0.2 g is added to this solution and is mixed until well dispersed.

The polyurethane with the silver sulfadiazine is then squeegeed onto paper or the like and dried in an oven at 70° C. for 10 minutes to flash off the solvent. The dried coated polyurethane is tested for elutable silver by placing a section of the coated paper under an ultraviolet lamp, adding a drop of water to a section of the paper and exposing the drop of water to the UV light for 30 to 60 minutes. It can be observed that the drop of water eventually turns gray; however, the time to turn the drop of water gray is significantly longer than the silver sulfonated polyurethane described in Example 1. This example suggests that the polyurethane is not as hydrophilic as Example 1 and does not as readily release the silver ion.

Coated sections of paper were then tested for antimicrobial activity by NAMSA using the Dow 923 “Shake-flask” test which involves shaking the sample in a flask with staphylococcus aureus bacteria for 1 hour and then for 24 hours and measuring the amount of bacteria killed. Results indicate that at one hour, 96.56%, or essentially most of the bacteria were killed. The results at 24 hours indicate that 99.94% of the bacterial charge was killed confirming that this formulation is bactericidal but not as effective as Example 1.

### Example 3

Control samples consisting of the same paper with the polyurethane binder alone, i.e., without the silver, killed 45.95% of the bacteria in one hour and 99.94% in 24 hours. An additional control sample of just the bottle alone showed a 38.89% reduction in bacteria at 24 hours. These controls indicate that the bottle as well as the paper with polyurethane coating are both somewhat bactericidal but not as much as the silver-treated samples of Examples 1 and 2.

### Conclusions Regarding Examples 1-3:

Any polymer can be used as the binder or carrier for the silver ion, such as polyurethane, polyolefin, silicone rubber, natural rubber, polyvinyl chloride, polyamide, polyester, cellulose, acetate, etc. as long as the polymer can be dissolved in a solvent or dispersed as a latex in a solvent. However, it is preferred that the polymer be somewhat hydrophilic to provide an aqueous medium for the silver ion to migrate towards the microbe. Preferred hydrophilic polymers include hydrophilic polyurethane, hydrogels such as poly(2-hydroxyethyl methacrylate), polyacrylamide, polyvinylpyrrolidone, etc. More preferred is hydrophilic polyurethane that can bind a metal cation such as silver. The sulfonated polyurethanes described in the above examples are such polyurethanes. Sulfonated hydrogels may also function in this capacity.

Metal ions other than silver (e.g. zinc) can be used as the antimicrobial agent. Silver is preferred because it is the most efficient of the metal ions for antimicrobial purposes.

In the above examples, it is preferable that polymer solutions of 0.1% to 45% be used. Polymer solutions with higher solids content are difficult to dissolve and difficult to mix. 5% to 15% solids is the most preferred range.

Although the solvent system of 20%/80% DMA/THF was used in Example 1 and 2, alternate solvents can be used to dissolve polyurethane such as m-pyrol, dimethylformamide, dimethylacetamide, dimethyl sulfonamide, mixtures of the above, mixtures of the above with swelling solvents such as diethyl ether, tetrahydrofuran, xylene, toluene etc. and the like. DMA/THF is preferred due to the ease of handling.

The concentration of acetic anhydride and sulfuric acid is equimolar. These chemicals combine in situ to sulfonate the polyurethane. The amount of sulfonation is controlled by the ratio of acetic anhydride/sulfuric acid to polymer. A suitable range of acetic anhydride/sulfuric acid:polyurethane, in mL/mL:g is 21/12.5:1 to 21/12.5:100. It was found empirically that about 21 ml of acetic anhydride and 12.5 ml of concentrated sulfuric acid to a solution with 10 g polyurethane provides a good balance of hydrophilicity to tensile strength. Too many sulfate groups on the polyurethane lower the tensile strength. Too few do not readily produce hydrophilic polyurethane.

The sulfonation concentration of 2% of solids content was described in Example 1. Other samples made at 0.5%, 10% and 20% also functioned as desired. However high loading of silver is unnecessarily expensive. Nevertheless, too low a loading may deplete the reservoir of available silver too quickly (i.e., in days rather than months or years). A concentration of 2% was selected as a rational intermediate concentration.

The concentration of silver sulfadiazine in Example 2 was 2% in respect to solids. Acceptable ranges are 0.1% to 20% for the same reasons as discussed in the previous paragraph.

The solutions described herein can be used to coat virtually any kind of paper. According to methods of the invention, it is expected that the antimicrobial solutions be used to coat paper used in sending mail such as envelopes and note paper. It is also expected that the antimicrobial solutions be used to coat financial instruments and paper currency which might be used by a terrorist to spread disease. Such solutions can also be mixed with printing ink for application on a paper web, another paper product, or another printed product.

### Example 4

The antimicrobial solution of Example 1 is prepared and is squeegeed onto both sides of a U.S. one dollar bill. The dollar bill is dried in an oven at 70° C. for 10 minutes to flash off the solvent. The coated dried dollar bill exhibits the same antimicrobial activity as the paper in Example 1.

Paper coated with the antimicrobial solution of the invention can be imprinted using offset printing, silkscreen printing, letterpress, rotogravure, flexible printing, liquid lamination, or coating.

The antimicrobial solutions may be applied to other products as described or by spraying, dipping, etc. According to the methods of the invention, it is also expected that the antimicrobial solution be applied to medical products such as surgical tools and implantable medical devices. If the medical device is polymeric, the antimicrobial agent can be applied as described in Example 4.

### Example 5

A polymeric medical device such as a catheter is sulfonated and rendered antimicrobial as follows.

A sulfonating solution is prepared with 93.3 ml 2-propanol, 4.2 ml acetic anhydride and 2.5 ml of concentrated sulfuric acid (added slowly). The solution is heated from room temperature to as high as the boiling point of the solvent; 60° C.±3° C., preferably with stirring. This sulfonating solution can be prepared in solvents other than 2-propanol, such as water, hexane, heptane, alcohols, etc., as long as the acetic anhydride and sulfuric acid are capable of dissolving in the solvent and the solvent is capable of wetting the polymer. 2-propanol is preferred for this reason. The ratio of 4.2 ml of acetic anhydride to 2.5 ml of sulfuric acid is selected so as to be a 1:1 molar ratio with the concentrated sulfuric acid.

The polymeric medical device is immersed in the above solution for 0.1 second to as long as 30 minutes; 10 seconds to 10 minutes is preferred. The device is removed and rinsed in deionized water for 1 to 30 minutes, 1 to 2 minutes with agitation is preferred. Ammonium hydroxide can be added to the deionized water to bring the pH back to neutral if necessary. The sulfonated polymeric device can be dried and stored, or it can immediately be rendered antimicrobial in the following manner.

A 2% silver nitrate solution is prepared by adding 2 g of silver nitrate to 100 ml of 2-propanol. The sulfonated polymeric device is immersed in this solution for 1 to 300 minutes; 30 minutes is preferred. The device is then rinsed in water and dried. The silver ion ionically bonds to the sulfate groups on the polymer. The concentration of silver nitrate can be between 0.01% and 20%. For economic reasons 0.1% to 2% is used. The solvent for the silver nitrate is 2-propanol; however, any solvent capable of dissolving silver nitrate and wetting the polymer can be used such as water, alcohols, etc.

An alternative method of producing a medical device according to the invention is demonstrated in Example 6.

### Example 6

A polymer solution is made by dissolving 10 g of an aromatic polyether urethane such as Dow Chemical's Pellethane™ 2363 75D in 90 g dimethyl acetamide (DMA) at 70° C. with mixing for 3 hours.

The polyurethane is sulfonated and rendered hydrophilic by adding 21 ml of acetic anhydride and 12.5 ml of concentrated sulfuric acid to the polyurethane solution while it is being vigorously mixed. After the exothermic reaction subsides, the hydrophilic urethane is poured into a blender filled with water where the polyurethane is precipitated and chopped into small particles under agitation. The particulate slurry is poured through a wire sieve to remove the particles and rinsed repeatedly with water until the pH of the solution is between 4 and 8. The precipitated sulfonated polyurethane is then dried for 3 hours in an oven at 70° C.

The dried sulfonated polyurethane (10 g) is then redissolved in 90 g DMA and then reacted with silver nitrate by adding 0.2 g (2% by weight of polymer) of silver nitrate to the sulfonated polyurethane solution. The solution is again precipitated and chopped into particles by pouring the solution into a blender containing water. The particles are rinsed repeated in water and then dried in a vacuum oven overnight at 70° C.

The dried particles containing silver, bound to sulfate groups on a polyurethane, are thermoplastic and can readily be extruded, injection molded, compression molded, or solvent cast into medical devices, and the like, using standard plastic processing equipment well known to people versed in the art of processing plastics.

In this manner, for example, catheters containing silver ion can be extruded directly without going through a second procedure.

Preferred materials for the catheter (or other polymeric medical device) include polyurethane, polyolefin, polyester, polyamide (Nylon and the like), polyimide and any other polymer capable of being sulfonated with the above reactants. The presently preferred polymer is polyurethane.

Polymeric medical devices that can benefit from antimicrobial activity include catheters, ports, scopes (endoscopes and the like) implantable devices in general, such as stents, vascular grafts, hip and knee acetabular joints, pacer lead insulators, spinal disks, sutures, stent grafts, etc.

As mentioned above, non-polymeric medical devices can be treated using the polymeric solutions described in Examples 1, 2 and 6.

### Example 7

Dried sulfonated polyurethane containing silver ion made according to Example 6 is dissolved in tetrahydrofuran at 5% solids content and squeegee-coated onto paper and flashed dried, thereby rendering the surface of the paper antimicrobial. Coatings made in this manner are similar to those described in Example 1. However, the dried polymer has a longer shelf life and is less expensive to inventory as compared to lacquers, and is therefore generally preferred.

In the examples above, oligodynamic metals (preferably silver) are ionically bound to a sulfonated and hydrophilic polymer (preferably a polyurethane that is sulfonated and rendered hydrophilic by adding acetic anhydride and sulfuric acid to a polyurethane solution while it is being vigorously mixed). The sulfonate of the sulfonated polymer is the counter-ion to the metal. As sulfonated polyurethane is hydrophilic but not totally soluble in water, water-soluble sulfonated polystyrene or copolymers of sulfonated polystyrene with maleic acid may be utilized as the polymer containing the sulfonate counter-ion. Thus, sulfonated polyurethane or sulfonated polystyrene, or mixtures thereof can be used interchangeably.

By adding one or more organic acids to the sulfonated polymer mixture, the total concentration of metals in the polymer mixture can be reduced significantly while maintaining or even enhancing antimicrobial activity. There seems to be a synergy amongst the chemicals that enhances their performance. Examples of organic acids include citric acid, maleic acid, ascorbic acid, salicyclic acid, acetic acid, formic acid and the like. In addition to the organic acids, other mildly acidic acids can also be used in this cocktail such as boric acid, dioctylborate, and the like.

### Example 8

Dried sulfonated polyurethane made according to Example 6 is dissolved in a solution and mixed with one or more oligodynamic metal compositions, one or more organic acids, and possible one or more non-organic acids. A preferred polyurethane is a polyethylene oxide based aromatic polyurethane that when sulfonated becomes water soluble. Alternatively, a water soluble sulfonated polystyrene or copolymers of sulfonated polystyrene with maleic acid may be substituted for the sulfonated polyurethane. When such water soluble polymers are used, the oligodynamic metal composition(s), organic acid(s), and non-organic acid(s) are water soluble such that the mixture readily dissolves in a water solution. Alternatively, the mixture can be dissolved in a solvent (e.g., m-pyrol, dimethylformamide, dimethylacetamide, dimethyl sulfonamide, mixtures of the above, mixtures of the above with swelling solvents such as diethyl ether, tetrahydrofuran, xylene, toluene etc. and the like).

Table 1 shows five experiments using various concentrations of acids and metals that are mixed and reacted to the sulfonated polymer carrier (showing actual amounts used and percentages (w/w))

| | Exp 1 | Exp | Exp 2 | Exp | Exp 3 | Exp | Exp 4 | Exp | Exp 5 | Exp |
|---|---|---|---|---|---|---|---|---|---|---|
| Chemical | (g) | 1% | (g) | 2% | (g) | 3% | (g) | 4% | (g) | 5% |
| AgNO₃ | 0.03 | 0.40 | 0.03 | 0.40 | 0.03 | 0.42 | 0.01 | 0.10 | 0.143 | 0.143 |
| Cu₂(NO₃)₂ | | 0.00 | 0.02 | 0.26 | 0.02 | 0.27 | 0.02 | 0.20 | 0.093 | 0.093 |
| Zn(NO₃)₂ | | 0.00 | 0.02 | 0.26 | 0.02 | 0.27 | 0.02 | 0.20 | 0.093 | 0.093 |
| Dioctylborate | 0.12 | 1.47 | 0.12 | 1.46 | 0.12 | 1.54 | | 0.00 | | |
| Acetic Acid | 0.05 | 0.62 | 0.05 | 0.62 | 0.05 | 0.65 | | 0.00 | | |
| Citric Acid | 0.05 | 0.62 | 0.05 | 0.62 | 0.05 | 0.65 | | 0.00 | 0.224 | 0.224 |
| Maleic Acid | 0.05 | 0.62 | 0.05 | 0.62 | 0.05 | 0.65 | | 0.00 | 0.224 | 0.224 |
| Boric Acid | 0.05 | 0.62 | 0.05 | 0.62 | 0.05 | 0.65 | | 0.00 | 0.224 | 0.224 |
| S. Polyurethane | | 0.00 | 0.07 | 0.87 | 0.07 | 0.95 | | 0.00 | 0.055 | 0.055 |
| Dimethyl | | 0.00 | | 0.00 | 7.20 | 93.94 | | 0.00 | | 0.00 |
| Acetamid | | | | | | | | | | |
| Water | 7.70 | 95.65 | 7.60 | 94.27 | | 0.00 | 10.40 | 99.51 | 98.94 | 98.94 |
| Total | 8.05 | 100.00 | 8.06 | 100.00 | 7.66 | 100.00 | 10.45 | 100.00 | 100 | 100 |

Testing of each mixture was performed as follows: Agar plates are inoculated with yeast (S. Cervecae) and dried. Looking at Experiment 5, for example, the cocktail was diluted 1:50 (˜2% concentration) with water and sprayed onto the agar plate containing the yeast and then placed in an incubator for 48 hours. After 48 hours the plates were examined and it was shown that the yeast cells where killed where the cocktail was applied. A similar experiment was conducted including filter paper or wood chips coated with the cocktail and dried. These coated samples were then placed on the yeast-inoculated agar and incubated for 48 hours and subsequently re-sprayed with more yeast and re-inoculated and re-incubated. This re-streaking was repeated numerous times with subsequent kills of the yeast cells in the sprayed area thereby demonstrating that the formulation on the paper or wood has longevity.

All of the mixtures provided the desired antimicrobial effects; however, the best kill was achieved with Experiment #5. Note, for example that in Experiment #2, the amount of water added was 94.27%, implying that the solids content was 5.63%. It was also found that the solids content can be varied between 0.0001% and 20% and give acceptable kills with the better kills provided by the higher solids content.

In the specific experiments of Table 1 were provided with divalent metals; however, monovalent or multivalent metals can also be used. Also note that when the organic carboxylic acids are mixed with the sulfonated polymer and the oligodynamic metal composition, a competing reaction occurs where some portion of the metal will couple with the sulfonated polymer and another portion of the metal will couple with the organic carboxylic acid(s). In the case where the metal couples with the sulfonated polymer, the counter ion is the sulfonate group on the polymer. In the case where the metal couples with the organic carboxylic acid(s), the counter ion is the organic carboxylic acid. The result of this competing reaction will depend on the stoicheometry, relative affinity and strength of the ionic bond.

The mixture of chemicals can be dried and ground to a fine powder and commercialized as such. In this case, the user need only dilute the powder with water to the desired concentration and spray, dip or dropped onto the substance to be coated. The antimicrobial agents described above may also be dissolved in a water solution (or solvent solution) and added as part of an admixture during formation of the end product. For example, the admixture may be a pulp that is processed to form a paper product. This is described in more detail in the following example.

### Example 9

Paper was made from a pulp made from torn up scrap paper and tap water blended with a hand blender and then poured through a screen and dried in an oven. Added to this pulp was a concentration of 1 gram of the antimicrobial agent made according to Example 8 per 600 grams, 700 grams, 800 grams and 1200 grams of pulp preparation, respectively while keeping one control made with all pulp preparation with no antimicrobial agent. The antimicrobial agent was derived by dissolving a water soluble sulfonated polyurethane in a solution and adding one or more oligodynamic metal compositions, one or more organic acids, and possibly one or more non-organic acids as set for in Experiment 5?? of example 8. The mixture was dried and ground to a fine powder. The paper was made from each of these well mixed pulp and agent diluted solutions, and dried in an oven at 80 degrees centigrade. The prepared paper were then labeled as “control”, “1/600”, “1/700”, “1/800” and “1/1200” according to their agent/pulp dilution values, and four squares of approximate equal size were cut from each paper.

TEST #1: Two of the four squares were pushed securely onto a malt extract agar plate and then sprayed with yeast solution, left to dry and then incubated at 37 degrees centigrade for 48 hours.

TEST #2: Another malt extract agar plate was sprayed with yeast solution, left to dry and then the remaining two squares of prepared paper were pushed securely on top of the dried yeast on the agar plate, and then incubated at 37 degrees centigrade for 48 hours.

The plates from TEST #1 and TEST #2 were then removed making sure that the yeast had grown up enough to be visible on the agar plates. One square of paper of each concentration from the TEST #1 plate and from the TEST #2 plate was removed using sterile tweezers, and was replaced onto another fresh malt extract agar plate. Using a sterile needle, the surface of the remaining square of paper was scraped and re-streaked onto a fresh malt extract agar plate to see if any visible cells were remaining. As a positive control, yeast was streaked onto the middle of the agar plate, to use as a time reference to compare yeast growth. TEST #3 was the restreaking of the plates of TEST #1, while TEST #4 was the restreaking of the plates of TEST #2. The plates from TEST #3 and TEST #4 were then incubated at 37 degrees centigrade for 24 hours, where the positive control of the streaked yeast was visibly grown up. These are the results that were encountered:

| | Control | 1/600 | 1/700 | 1/800 | 1/1200 |
|---|---|---|---|---|---|
| TEST #1 | Growth not | Growth partly | Growth partly | Growth partly | Growth partly |
| | inhibited | inhibited | inhibited | inhibited | inhibited |
| | under square | under square | under square | under square | under square |
| TEST #2 | Growth not | Growth | Growth | Growth | Growth |
| | inhibited | inhibited | inhibited | inhibited | inhibited |
| | under square | under square | under square | under square | under square |
| TEST #3 | Re-streak | Re-streak did | Re-streak did | Re-streak did | Re-streak did |
| | grew | not grow | not grow | not grow | not grow |
| TEST #4 | Re-streak | Re-streak did | Re-streak did | Re-streak did | Re-streak did |
| | grew | not grow | not grow | not grow | not grow |

Other paper products made with other antimicrobial agents according to example 8 have also been tested in a similar manner to those described above in example 9. Such paper has also produced acceptable kill levels of yeast cells applied thereto. It is also contemplated that any of the other antimicrobial agents described herein will be suitable for use in a paper product

There have been described and illustrated herein antimicrobial agents, products incorporating said agents and methods of making the antimicrobial agents and products incorporating them. While particular embodiments of the invention have been described, it is not intended that the invention be limited thereto, as it is intended that the invention be as broad in scope as the art will allow and that the specification be read likewise. It will therefore be appreciated by those skilled in the art that yet other modifications could be made to the provided invention without deviating from its spirit and scope as so claimed.

1. An antimicrobial agent comprising:
an oligodynamic metal ion reacted with an acidic group counter-ion of a hydrophilic polymer containing a counter-ion bound to it, said hydrophilic polymer controlling a sustained release of the metal ion, and said hydrophilic polymer including polyurethane.

2. An antimicrobial agent according to claim 1, wherein:
said oligodynamic metal ion is derived from a metal selected from the group consisting of Ag, Au, Pt, Pd, Ir, Cu, Sn, Sb, Bi and Zn.

3. An antimicrobial agent according to either of claims 1 or 2, wherein:
said oligodynamic metal ion is Ag+.

4. (canceled)

5. (canceled)

6. An antimicrobial agent according to any previous claim, wherein:
said hydrophilic polymer is sulfonated polyurethane.

7. (canceled)

8. A paper product comprising:
paper having a surface coated with an antimicrobial having an oligodynamic metal ion reacted with a hydrophilic polymer containing a counter-ion which controls a sustained release of the metal ion.

9. A paper product according to claim 8, wherein:
said oligodynamic metal ion is derived from a metal selected from the group consisting of Ag, Au, Pt, Pd, Ir, Cu, Sn, Sb, Bi and Zn.

10. A paper product according to claim 8, wherein:
said oligodynamic metal ion is Ag+.

11. A paper product according to claim 8, wherein:
said hydrophilic polymer includes a polymer selected from the group consisting of polyurethane, polyamines, cellulose, cellulose acetate, triacetate, polyester, hydrogels, and polyolefins.

12. A paper product according to claim 8, wherein:
said hydrophilic polymer includes polyurethane.

13. A paper product according to claim 12, wherein:
said hydrophilic polymer is sulfonated polyurethane.

14. A paper product according to claim 8, wherein:
said oligodynamic metal ion is Ag+, and
said hydrophilic polymer is sulfonated polyurethane.

15. A paper product according to claim 8, wherein:
said paper is printed currency.

16. An improvement in a medical device having a surface, said improvement comprising:
said surface being active with an antimicrobial agent according to any of claims 1-3.

17-20. (canceled)

21. A medical device according to claim 16, wherein:
said hydrophilic polymer is sulfonated polyurethane.

22. (canceled)

23. A medical device according to claim 16 or 21, wherein:
said medical device is selected from the group consisting of catheters, ports, scopes, implantable devices, stents, vascular grafts, hip and knee acetabular joints, pacer lead insulators, spinal disks, sutures, and stent grafts.

24. A medical device having a polymeric outer surface rendered hydrophilic and reacted with oligodynamic metal ion.

25. A method of making an antimicrobial coating suitable for coating paper to impart the paper with antimicrobial properties, said method comprising:
a) rendering a polymer hydrophilic;
b) reacting the hydrophilic polymer with an oligodynamic metal ion; and
c) adding the hydrophilic polymer and oligodynamic metal ion to a solvent suitable for coating paper.

26. A method according to claim 25, wherein:
said oligodynamic metal ion is derived from a metal selected from the group consisting of Ag, Au, Pt, Pd, Ir, Cu, Sn, Sb, Bi and Zn.

27. A method according to claim 25, wherein:
said oligodynamic metal ion is Ag+.

28. A method according to claim 25, wherein:
said polymer is a polymer selected from the group consisting of polyurethane, polyamines, cellulose, cellulose acetate, triacetate, polyester, hydrogels, and polyolefins.

29. A method according to claim 25, wherein:
said polymer is polyurethane.

30. A method according to claim 29, wherein:
said hydrophilic polymer is sulfonated polyurethane.

31. A method according to claim 25, wherein:
said oligodynamic metal ion is Ag+, and
said hydrophilic polymer is sulfonated polyurethane.

32. A method according to claim 25, wherein:
said solvent is selected from the group consisting of m-pyrol, dimethylformamide, dimethylacetamide, dimethyl sulfonamide, diethyl ether, tetrahydrofuran, xylene, and toluene.

33. A method according to claim 25, wherein:
said solvent is 20%/80% DMA/THF.

34. A method for imparting paper with antimicrobial properties, said method comprising:
a) reacting a hydrophilic polymer with an oligodynamic metal ion;
b) adding the hydrophilic polymer and oligodynamic metal ion to a solvent suitable for coating paper;
c) applying the resulting mixture to the paper; and
d) drying the paper to remove the solvent.

35. A method according to claim 34, wherein:
said oligodynamic metal ion is derived from a metal selected from the group consisting of Ag, Au, Pt, Pd, Ir, Cu, Sn, Sb, Bi and Zn.

36. A method according to claim 34, wherein:
said oligodynamic metal ion is Ag+.

37. A method according to claim 34, wherein:
said hydrophilic polymer includes a polymer selected from the group consisting of polyurethane, polyamines, cellulose, cellulose acetate, triacetate, polyester, hydrogels, and polyolefins.

38. A method according to claim 34, wherein:
said hydrophilic polymer includes polyurethane.

39. A method according to claim 34, wherein:
said hydrophilic polymer is sulfonated polyurethane.

40. A method according to claim 34, wherein:
said oligodynamic metal ion is Ag+, and
said hydrophilic polymer is sulfonated polyurethane.

41. A method according to claim 34, wherein:
the paper is printed currency.

42. A method of producing a water-soluble antimicrobial agent comprising:
i) dissolving a water soluble polymer in water to make a solution; and
ii) adding a water soluble metal composition and at least one organic acid to said solution, wherein oligodynamic metal ions of said metal composition reacts with counter-ions of said polymer such that said metal ions are bound to corresponding counter-ions, and said polymer controls a sustained release of said metal ions.

43. A method according to claim 42, further comprising:
iii) adding at least one non-organic acid to said solution.

44. A method according to claim 42, wherein:
said water soluble polymer comprises a sulfonated polymer.

45. A method according to claim 44, wherein:
said counter-ions are sulfonate ions of said sulfonated polymer.

46. A method according to claim 44, wherein:
said sulfonated polymer comprises a sulfonated polyurethane.

47. A method according to claim 44, wherein:
said sulfonated polymer comprises a sulfonated polystyrene.

48. A method according to claim 42, wherein:
said organic acid comprises at least one of: acetic acid, citric acid, maleic acid, ascorbic acid, salicyclic acid, and formic acid.

49. A method according to claim 43, wherein:
said non-organic acid comprises at least one of: boric acid and dioctylborate.

50. A method according to claim 42, further comprising:
drying the solution resulting from the step ii) into a solid form, and grinding the solid form into a powder.

51. A method according to claim 43, further comprising:
drying the solution resulting from the step iii) into a solid form, and grinding the solid form into a powder.

52. A method according to claim 42, wherein:
said water soluble metal composition includes a metal selected from the group consisting of Ag, Au, Pt, Pd, Ir, Cu, Sn, Sb, Bi and Zn.

53. A method according to claim 42, wherein:
said water soluble metal composition includes at least one composition selected from the group consisting of silver nitrate (AgNO₃), copper nitrate (Cu(NO₃)₂), and zinc nitrate (Zn(NO₃)₂).

54. A method according to claim 42, wherein:
said oligodynamic metal ion is Ag+.

55. An antimicrobial agent comprising:
a water soluble polymer, at least one organic acid, and oligodynamic metal ions which react with counter-ions of said polymer such that said metal ions are bound to corresponding counter-ions, and said polymer controls a sustained release of said metal ion.

56. An antimicrobial agent according to claim 55, further comprising:
a non-organic acid.

57. An antimicrobial agent according to claim 56, wherein:
said water soluble polymer comprises a sulfonated polymer.

58. An antimicrobial agent according to claim 57, wherein:
said counter-ions are sulfonate ions of said sulfonated polymer.

59. An antimicrobial agent according to claim 57, wherein:
said sulfonated polymer comprises a sulfonated polyurethane.

60. An antimicrobial agent according to claim 57, wherein:
said sulfonated polymer comprises a sulfonated polystyrene.

61. An antimicrobial agent according to claim 55, wherein:
said organic acid comprises at least one of: acetic acid, citric acid, maleic acid, ascorbic acid, salicyclic acid, and formic acid.

62. An antimicrobial agent according to claim 56, wherein:
said non-organic acid comprises at least one of: boric acid and dioctylborate.

63. An antimicrobial agent according to claim 55, wherein:
said oligodynamic metal ions are Ag+.

64. An antimicrobial agent according to claim 55, wherein:
said oligodynamic metal ions are selected from the group consisting of Au+, Pt+, Pd+, Ir+, Cu²⁺, Sn+, Sb+, Bi+ and Zn²⁺.

65. A method for inhibiting microbial growth on a target comprising:
providing a water soluble antimicrobial agent comprising a water soluble polymer, at least one organic acid, and oligodynamic metal ions which react with a counter-ions of said polymer such that said metal ions are bound to corresponding counter-ions, and said polymer controls a sustained release of said metal ions;
dissolving said water soluble antimicrobial agent in a water solution; and
applying said water solution to said target.

66. A method according to claim 65, wherein:
the water solution is applied to said target as a coating or film by spraying or dipping.

67. A method for inhibiting microbial growth on a target comprising:
providing a water soluble antimicrobial agent comprising a water soluble polymer, at least one organic acid, and oligodynamic metal ions which react with a counter-ions of said polymer such that said metal ions are bound to corresponding counter-ions, and said polymer controls a sustained release of said metal ions;
dissolving said water soluble antimicrobial agent in a water solution; and
adding said water solution as part of an admixture during formation of said target.

68. A method according to claim 67, wherein:
said admixture comprises a pulp slurry that is processed to form a web of paper.

69. An antimicrobial agent comprising:
a plurality of different oligodynamic metal ions which react with counter-ions of a polymer such that said metal ions are bound to corresponding counter-ions, said polymer controlling a sustained release of said metal ions, wherein said plurality of different oligodynamic metal ions include Ag+, Cu²⁺ and Zn²⁺.

70. An antimicrobial agent according to claim 69, wherein:
wherein said plurality of different oligodynamic metal ions consist of Ag+, Cu²⁺ and Zn²⁺.

71. An antimicrobial agent according to claim 69, wherein:
said counter-ions comprise hydrophilic groups of said polymer.

72. An antimicrobial agent according to claim 71, wherein:
said hydrophilic groups include sulfates and organic acids.

73. An antimicrobial agent according to claim 72, wherein:
said organic acids comprise at least one carboxylic acid (preferably acetic acid, formic acid, citric acid, maleic acid, ascorbic acid, salicyclic acid)

74. An antimicrobial agent according to claim 72, further comprising:
at least one inorganic acid (preferably boric acid, dictylborate).

75. An antimicrobial agent according to claim 69, wherein:
the Ag+, Cu²⁺ and Zn²⁺ ions are reacted to said polymer by dissolving in a water solution a water soluble polymer and water soluble metal compositions comprising Ag, Cu and Zn.

76. An antimicrobial agent according to claim 75, wherein:
said water soluble metal compositions include silver nitrate (AgNO₃), copper nitrate (Cu(NO₃)₂), and zinc nitrate (Zn(NO₃)₂).

77. An antimicrobial agent according to claim 76, wherein:
said water soluble metal compositions together comprise less than 0.4 percent by weight of the water solution.

78. An antimicrobial agent according to claim 77, wherein:
said water solution comprises the copper nitrate and the zinc nitrate each at a percent by weight that is 0.667 relative to the percent by weight of the silver nitrate.

79. An antimicrobial agent according to claim 69, wherein:
said polymer comprises a sulfonated polymer.

80. An antimicrobial agent according to claim 79, wherein:
said sulfonated polymer comprises a sulfonated polyurethane.

81. An antimicrobial agent according to claim 79, wherein:
said sulfonated polymer comprises a sulfonated polystyrene.

82. An antimicrobial agent comprising:
oligodynamic metal ions which react with counter-ions of a polymer such that said metal ions are bound to corresponding counter-ions, said counter-ions comprising hydrophilic groups which include sulfate groups and at least one organic acid, said polymer controlling a sustained release of said metal ions.

83. An antimicrobial agent according to claim 82, wherein:
said oligodynamic metal ions include at least one of Ag+, Cu²⁺ and Zn²⁺.

84. An antimicrobial agent according to claim 83, wherein:
wherein said oligodynamic metal ions consist of Ag+, Cu²⁺ and Zn²⁺.

85. An antimicrobial agent according to claim 82, wherein:
said at least one organic acid comprises at least one carboxylic acid (preferably selected from the group of acetic acid, formic acid, citric acid, maleic acid, ascorbic acid, and salicyclic acid)

86. An antimicrobial agent according to claim 82, further comprising:
at least one inorganic acid (preferably selected from the group of boric acid, and dictylborate).

87. An antimicrobial agent according to claim 82, wherein:
said oligodynamic metal ions are reacted to said polymer by dissolving in a water solution a water soluble sulfonated polymer, at least one water soluble metal composition comprising said oligodynamic metal ions, and said organic acid.

88. An antimicrobial agent according to claim 87, wherein:
said water soluble metal compositions include silver nitrate (AgNO₃), copper nitrate (Cu(NO₃)₂), and zinc nitrate (Zn(NO₃)₂).

89. An antimicrobial agent according to claim 87, wherein:
said water soluble sulfonated polymer comprises a water soluble sulfonated polyurethane.

90. An antimicrobial agent according to claim 87, wherein:
said water soluble sulfonated polymer comprises a water soluble sulfonated polystyrene.
